(19) United States
(12) Patent Application Publication
(10) Pub. No.: US 2006/0257323 A1
(43) Pub. Date: Nov. 16, 2006

(54) Preventing insufflation and injection modes of drug abuse

(76) Inventor: John C. Kulli, Hamlin, NY (US)

Correspondence Address: PETER I. LIPPMAN, 17900 MOCKINGBIRD LANE, RENO, NV, 89506, US

(21) Appl. No.: 11/406,886

(22) Filed: Apr. 19, 2006

Related U.S. Application Data
(60) Provisional application No. 60/676,865, filed on May 2, 2005.

(51) Int. Cl. A61K 9/44; G06Q 50/00

(52) U.S. Cl. 424/10.2; 705/2

(57) ABSTRACT

A drug-formulating method, a drug commercial-distribution method, and a drug formulation improve safety of a drug that is at risk for abuse—such as methylphenidate, or amphetamine, or an amphetamine-like central-nervous-system stimulant. The drug is formulated into a form (not a transdermal patch) that tends to deter conversion to powder; and in this form commercially distributed, e. g. wholesale or retail. Preferably that form is enclosed in a nontoxic capsule. Preferably the formulating includes dissolving or dispersing the drug into or onto a nontoxic carrier—for example a gel, e. g., methylcellulose, hydroxymethylcellulose, carbomer polymer, or other gelatinous pharmaceutical agent that is FDA-acceptable. Preferably the carrier is water-insoluble, to deter dissolving in water for injection. The carrier may be an oil or a solid—for example paper or other thin medium broadly extended in two dimensions, or a sponge or other medium having generally coarse cellular structure.

This application is based in part upon, and claims the priority benefit of, my provisional patent application 60/676,865 filed May 2, 2005—which is wholly incorporated by reference herein.

### BACKGROUND

Methylphenidate (Ritalin® and others) and amphetamine or amphetamine-like central nervous system stimulant drugs are widely prescribed in the U. S. and other countries for the treatment of attention-deficit disorder. Unfortunately, these drugs are frequently diverted for illicit use.

The commonest mode of abuse of methylphenidate is nasal insufflation (snorting). The methylphenidate tablet is ground into a fine dust and then rapidly snorted, thereby dispersing the drug powder widely onto the nasal mucosa where it is rapidly absorbed into the bloodstream.

This route of administration bypasses the stomach enzymes and the liver's detoxifying enzymes. As a result, the effect of insufflated methylphenidate far more rapid and potent than the effect of the same dose taken orally.

Insufflation of drugs intended for oral administration is dangerous for several reasons. First, the drugs are usually compounded with other chemical agents designed to improve the handling characteristics of the tablet.

These can include, according to the manufacturer, lactose, starch, polyethylene glycol, magnesium stearate, sucrose, talc, cellulose, mineral oil, and various dyes and conditioning agents. Although these agents are nontoxic when taken orally, they can have serious health effects when insufflated.

Second, because of the far more effective delivery of these drugs to the central nervous system, there is a real risk of toxic effects, particularly powerful neurological effects and changes in heart rate and rhythm.

Methylphenidate is easily prepared for insufflation by simply grinding a tablet into powder. Among college students this is frequently done by placing the tablet 11 (FIG. 1) in the depression in the bottom of a small aspirin bottle or other pill bottle 12, and crushing the tablet into fragments 11′ with a coin 13.

Often or usually, crushing continues until much of the pill has been reduced to a finer, inhalable powder 11″ (FIG. 3). This material is brought near nostrils 15 of the substance abuser, who then inhales to draw a stream 14 of the powder into the nostrils.

Methylphenidate is also abused by injection. This is accomplished after dissolving the crushed tablet in water, aided by heating the water. The resulting aqueous solution of methylphenidate is then injected.

A liquid preparation of methylphenidate exists commercially, but it is not formulated to discourage extraction. The liquid form is relatively much harder than the powder to administer, and therefore is disfavored, in some environments—particularly in elementary school, where accurate measurement of dosage of liquid medications is difficult or impossible. The liquid form also is susceptible to diversion by dilution.

Recently two new methylphenidate products have come onto the market. One is a so-called “chewable” Methylin® tablet, containing guar gum and pregelatinized starch.

Notwithstanding the use of the term “chewable”, these additives apparently do not actually impart a chewy texture to the medication, but rather merely bulk it up. A substance abuser can pulverize and then insufflate the entire preparation—guar, starch, methylphenidate, and any flavoring such as Aspartame® etc.

It appears from the official FDA “package insert” that no thought was given to antiinsufflation properties. In particular this tablet was not designed to prevent insufflation, but rather merely for patients (particularly young children) who are unable to swallow pills intact.

The other new Ritalin product is a transdermal patch. The patch itself is insufflation proof; however, for at least two reasons it does not solve the abuse problems described above.

First, it does not entirely replace or supplant tablet Ritalin, but instead leaves the latter on the market. Second, appearance of the patch makes it obvious to anyone who sees the device that the child or other patient uses Ritalin, subjecting the user to taunting, harassment, etc.

Wearing the patch under clothing cannot fully eliminate this problem. Junior-high-school and high-school boys typically are required in physical-education classes to undress completely to shower—thus revealing the patch and giving rise to the above-mentioned harassing or taunting behavior, perhaps in aggravated form.

It is possible or even likely that a child will remove the patch, to avoid such embarrassments—or to sell it to a peer who wants the sensation (“kick”) which the drug produces. Further, because it is visible, there is a significant risk that the patch will be taken from the patient by force—or, before being applied to the patient, stolen from the patient's purse, gym locker etc.

According to the U. S. Federal Drug Enforcement Administration, thirty to fifty percent of adolescents in drug treatment reported so-called “nonmedical” use of methylphenidate; the DEA has accordingly added methylphenidate to its list of “Drugs of Concern”. NIDA reports a study in 2004 showing that over five percent of high-school seniors abuse Ritalin, and as to the college level the Johns Hopkins Newsletter (April 2005) estimates that as many as one in five college students have used methylphenidate illegally.

Because prescription use of methylphenidate is both lawful and common, and prescriptions are easy to obtain, even the direct discovery of methylphenidate in drug screenings is of little value in identifying abusers. Throughout this document, where the term “methylphenidate” appears, to the extent appropriate to the context that term shall be understood to encompass any drug susceptible to insufflation or injection.

Thus the threat posed by this type of drug is extremely insidious—and yet it is a known killer. The prior art has failed to significantly address this problem, and thus has left great room for improvement.

### SUMMARY OF THE DISCLOSURE

The present invention provides exactly such improvement. In preferred embodiments of a first of its aspects or facets the invention is a method for improving the safety of a drug at risk for abuse.

The method includes the step of formulating the drug into a form, other than a transdermal patch, that tends to deter conversion to powder. It also includes the step of providing the drug for commercial distribution in that form.

The foregoing may represent a description or definition of the first aspect or facet of the invention in its broadest or most general form. Even as couched in these broad terms, however, it can be seen that this facet of the invention importantly advances the art.

In particular, by preparing the drug in a form that tends to deter conversion to powder, the invention bears significant potential for reducing the widespread abuse of the common drugs mentioned above. A marked reduction may be expected.

For the reason explained in the preceding section of this document, a transdermal patch (such as is already on the market) cannot effectively prevent the abuse problems mentioned above. Therefore the foregoing definition or description expressly excludes a transdermal patch.

Although the first major aspect of the invention thus significantly advances the art, nevertheless to optimize enjoyment of its benefits preferably the invention is practiced in conjunction with certain additional features or characteristics. In particular, preferably the method further includes the step of, before the marketing step, enclosing the drug, in said form, in a nontoxic capsule. Another preference is that be specifically methylphenidate, or amphetamine, or an amphetamine-like central-nervous-system stimulant.

Still another preference is that the formulating step be performed by dissolving or dispersing the drug into or onto a nontoxic carrier. If this basic preference is observed, then it is further preferable that the carrier be a gel. In this case it is still further preferred that the gel be selected from the group consisting of methylcellulose, hydroxymethylcellulose, carbomer polymer, and other gelatinous agents on an FDA list of acceptable pharmaceutical agents.

Other options for the carrier are that it be:
water-insoluble—whereby the formulating inhibits dissolving the drug in water for injection;
an oil;
a solid—in which case, alternative preferences are that the solid be in the form of paper or other thin medium broadly extended in two dimensions; or in the form of a sponge or other medium having a generally coarse cellular structure.

In preferred embodiments of its second major independent facet or aspect, the invention is a product or composition of matter—in a particular, a drug formulation for improving the safety of a drug at risk for abuse. The formulation includes the drug, in a form (other than a transdermal patch) that tends to deter conversion to powder. It also includes a capsule containing the drug in that form.

The foregoing may represent a description or definition of the second aspect or facet of the invention in its broadest or most general form. Even as couched in these broad terms, however, it can be seen that this facet of the invention importantly advances the art.

In particular, the benefits of this product aspect of the invention is closely related to those of the method aspect discussed above. These benefits particularly include reducing the availability of these drugs in conditions that are susceptible to abuse—and thus reducing injuries and deaths caused by such abuse.

Although the second major aspect of the invention thus significantly advances the art, nevertheless to optimize enjoyment of its benefits preferably the invention is practiced in conjunction with certain additional features or characteristics. In particular, preferably the formulation includes a solution or dispersion of the drug in or on a nontoxic carrier. Other preferences described above for the first aspect of the invention are also applicable here.

In preferred embodiments of its third major independent facet or aspect, the invention is a commercial distribution method for improving the safety of a drug at risk for abuse. The method includes the steps of obtaining the drug in a form, other than a transdermal patch, that tends to deter conversion to powder.

The method also includes the step of selling the drug in said form. The foregoing may represent a description or definition of the third aspect or facet of the invention in its broadest or most general form.

Even as couched in these broad terms, however, it can be seen that this facet of the invention importantly advances the art. In particular, the sale of commonly abused drugs in a pulverization-deterring form enlists the commercial process in the aid of reducing rates and severity of drug abuse.

Although the third major aspect of the invention thus significantly advances the art, nevertheless to optimize enjoyment of its benefits preferably the invention is practiced in conjunction with certain additional features or characteristics. In particular, preferably the selling step includes wholesale selling; it also preferably includes retail selling.

Another preference is that the form include a solution or suspension of the drug in or on a nontoxic carrier. If this preference is observed, then further preferences include obtaining the carrier:
as a gel, a solid or a liquid; or
as an oil; or
in the form of a paper, or other thin material broadly extended in two dimensions; or a sponge or other material having generally coarse cellular structure.

The foregoing features and advantages of the invention will be more fully appreciated from the following Detailed Description, considered together with the accompanying drawings—of which:

### BRIEF DESCRIPTION OF THE DRAWINGS

FIG. 1 is a perspective or isometric view showing an inverted pill bottle with a pill placed thereon, as commonly done in abuse of Ibuprofen® or the like;

FIG. 2 is a like view showing a further stage in the abuse process;

FIG. 3 is another like view showing a final step; and

FIG. 4 is a flow chart representing very schematically the steps in practice of my invention.

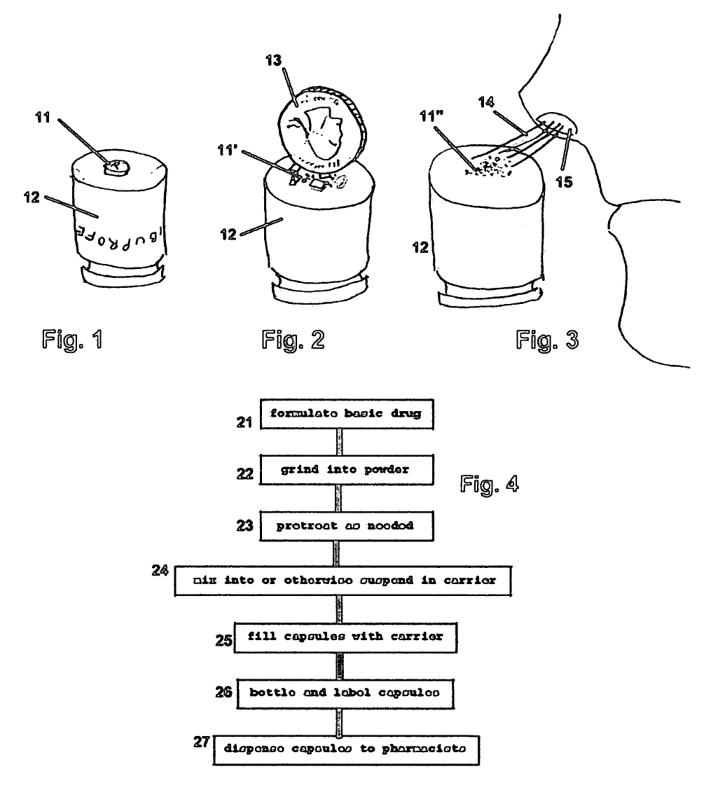

### DETAILED DESCRIPTION OF THE PREFERRED EMBODIMENTS

Methylphenidate for oral administration can be reformulated into a capsule containing the active drug, methylphenidate, and whatever of the inert ingredients that might be of value in this novel formulation, all dispersed or dissolved in a carrier. The carrier is chosen or formulated to deter insufflation and other modes of abuse mentioned above.

Thus it may be a gel such as methylcellulose, hydroxypropylmethylcellulose, sodium carboxymethylcellulose or a carbomer polymer such as Carbopol® 934P. Alternatively the carrier may be a liquid, in which the drug is suspended or preferably dissolved; or a solid.

A particularly advantageous liquid is one—such as (merely by way of example) an oil—from which the drug is awkward or difficult to remove without special equipment or training, or preferably both. Liquids from which the drug can be recovered simply by e. g. drying, evaporation or settling, or common centrifugation, should not be used.

Gel is more advantageous than liquid in general, but some particular liquids may be very effective. As to solid-phase carriers, the drug should be adhered to (as for example by gluing) or embedded within a solid—such as paper, sponge, or a gelatinous etc. confection or pastry—that is not easy to grind.

The carrier should be chosen to avoid interfering with the desired pharmacological properties of the drug, when the carrier and drug together are ingested in the approved usual way—e. g. in most cases orally. This is particularly noteworthy in the case of a solid carrier such as paper, sponge, etc., since the selected solid must be one that can be chewed, or in any event harmlessly swallowed and digested, notwithstanding its incompatibility with mechanical grinding.

If the drug is dispersed as small particles throughout or on the carrier, the combination is called a two-phase system. If the drug appears to be completely dissolved in the carrier, the resulting clear or colored formulation is a one-phase system.

The most difficult manufacturing problem with a gel formulation is ensuring that the drug is dispersed evenly, to a reasonable degree, throughout the gel and substantially without clumping. This may be accomplished by:
smoothly sifting the powdered drug into the rapidly stirring gel,
using a blender to completely homogenize the powder/gel mixture, or
making a smooth paste of the drug with a water-miscible agent such as an alcohol or propylene glycol.

Methylphenidate itself is freely soluble in water, and therefore should easily make a one-phase gel with methylcellulose or hydroxyethylcellulose, both of which are commonly used pharmaceutical compounding agents. The several carrier ingredients mentioned above include many that are already FDA approved; thus, approval requirements for at least initial practice of this invention should be minimal.

Bioavailability for gels should not be a problem, because many gels suitable for use in practicing this invention are inert. Cellulose gels are routinely used as inert thickeners in various food products.

In any event, the carrier with the drug in it is placed or molded into a capsule of, ideally, a commonly used type of encapsulating material—or a material with closely related characteristics. Such capsules, as is well known, in common use dissolve rapidly to release the drug in the stomach—and this should be the case in practice of the present invention as well.

Given suitable choice of carrier, the resulting combination of the drug and carrier, enclosed in or molded as a capsule, should have essentially the same pharmacological characteristics as the tablet form of the capsule.

Snorting of the capsule contents will, however, be substantially impossible. Physical properties of the gel, a sticky semifluid, prevent breaking the drug into a fine aerosol and thus block its wide dispersion onto the nasal mucosa. Separating drug from carrier, though possible in a specially equipped lab, would be difficult otherwise.

An object of this invention, as will be clear from the foregoing discussion, is simply to deter physical or chemical separation of the drug from the carrier. To those skilled in this field, however, it will also be apparent that it is not absolute deterrence that is required—to make practice of my invention extremely valuable societally —but rather only a meaningful tendency to deter.

Absolute deterrence could be desirable, but achieving that degree of abuse obstruction might be inordinately expensive or complex. Some people intent upon abusing or facilitating abuse of drugs may have access to highly advanced physical or chemical separation facilities and abilities, such as are found in industrial or graduate-level laboratories. Extreme measures needed to preclude separation by such individuals may be impractical or produce new adverse consequences.

Rather the degree of deterrence particularly sought through practice of the present invention is that which obstructs simple home-based methods such as are popular among casual abusers, e. g., as noted above, college students. The very great majority of these people have neither access to special means of separation nor an inclination to expend large sums of money to purchase such services.

To make the formulation resistant to injection abuse, the drug should be dispersed into a gel or other material that is not soluble in water as part of a two-phase system. Given this precaution, the capsule contents will not be easily converted into injection form.

Thus my invention is advantageously practiced by the steps comprising: formulating 21 (FIG. 4) the basic drug, grinding 22 the formulated drug into powder, applying any pretreatment 23 that may be needed, and mixing or otherwise suspending 24 the drug into a carrier. Thereafter, the invention may comprise the further steps of filling 25 capsules with the drug-containing carrier, bottling and labeling the capsules 26, and dispensing the capsules to pharmacists 27.

The foregoing disclosure is intended to be merely exemplary and not to limit the scope of the invention—which is defined by the appended claims.

I claim:

1. A method for improving the safety of a drug at risk for abuse; said method comprising the steps of:
formulating the drug into a form, other than a transdermal patch, that tends to deter conversion to powder; and
providing the drug for commercial distribution in said form.

2. The method of claim 1, further comprising the step of:
before the marketing step, enclosing the drug, in said form, in a nontoxic capsule.

3. The method of claim 1, wherein:
the drug is specifically methylphenidate, or amphetamine, or an amphetamine-like central-nervous-system stimulant.

4. The method of claim 1, wherein:
the formulating step is performed by dissolving or dispersing the drug into or onto a nontoxic carrier.

5. The method of claim 4, wherein:
the carrier is a gel.

6. The method of claim 5, wherein:
the gel is selected from the group consisting of methylcellulose, hydroxymethylcellulose, carbomer polymer, and other gelatinous agents on an FDA list of acceptable pharmaceutical agents.

7. The method of claim 1, wherein:
the carrier is water-insoluble;
whereby the formulating inhibits dissolving the drug in water for injection.

8. The method of claim 1, wherein:
the carrier is an oil.

9. The method of claim 1, wherein:
the carrier is a solid.

10. The method of claim 9, wherein:
the solid is in the form of paper or other thin medium broadly extended in two dimensions.

11. The method of claim 9, wherein:
the solid is in the form of a sponge or other medium having a generally coarse cellular structure.

12. A drug formulation for improving the safety of a drug at risk for abuse; said formulation comprising:
the drug, in a form, other than a transdermal patch, that tends to deter conversion to powder; and
a capsule containing the drug in said form.

13. The formulation of claim 12, wherein:
said form comprises a solution or dispersion of the drug in or on a nontoxic carrier.

14. A commercial distribution method for improving the safety of a drug at risk for abuse; said method comprising the steps of:
obtaining the drug in a form, other than a transdermal patch, that tends to deter conversion to powder; and
selling the drug in said form.

15. The method of claim 14, wherein:
the selling step comprises wholesale selling.

16. The method of claim 14, wherein:
the selling step comprises retail selling.

17. The method of claim 14, wherein:
said form comprises a solution or suspension of the drug in or on a nontoxic carrier.

18. The method of claim 17, wherein:
the carrier is a gel, a solid or a liquid.

19. The method of claim 17, wherein:
the carrier is an oil.

20. The method of claim 17, wherein:
the carrier is in the form of a paper, or other thin material broadly extended in two dimensions; or a sponge or other material having generally coarse cellular structure.
